(19) United States
(12) Patent Application Publication
(10) Pub. No.: US 2010/0143976 A1
(43) Pub. Date: Jun. 10, 2010

(54) PROCESS OF INCREASING CELLULAR PRODUCTION OF BIOLOGICALLY ACTIVE COMPOUNDS

(75) Inventors: J. Mark WEBER, Chicago, IL (US); Andrew R. Reeves, Chicago, IL (US); Igor A. Brikun, Forest Park, IL (US); William Henry Cernota, Chicago, IL (US)

Correspondence Address: QUARLES & BRADY LLP, 411 E. WISCONSIN AVE., SUITE 2040, MIWAUKEE, WI, 53202-4497, US

(73) Assignee: FERMALOGIC, Inc., Chicago, IL (US)

(21) Appl. No.: 12/614,976

(22) Filed: Nov. 9, 2009

Related U.S. Application Data
(63) Continuation-in-part of application No. 12/468,456, filed on May 19, 2009.
Application No. 12/468,456 is a division of application No. 10/637,159, filed on Aug. 8, 2003, now Pat. No. 7,638,306.
Application No. 10/637,159 is a continuation-in-part of application No. 11/466,364, filed on Aug. 22, 2006.
(60) Provisional application No. 60/710,412, filed on Aug. 22, 2005.

(51) Int. Cl. C12P 19/60; C12N 1/21; C12N 1/20

(52) U.S. Cl. 435/75; 435/252.3; 435/253.6

(57) ABSTRACT

A method of increasing production of a methylmalonyl-CoA derivative in a bacterium is disclosed that includes providing a bacterium that produces a methylmalonyl-CoA derivative in which methylmalonyl-CoA mutase function has been inhibited and culturing the bacterium in a medium containing an effective amount of methylmalonyl-CoA or a precursor thereof to increase the production of the methylmalonyl-CoA derivative by the bacterium. The production of the methylmalonyl-CoA derivative is increased when compared to production of the methylmalonyl-CoA derivative by the bacterium when cultured in a medium without the effective amount of methylmalonyl-CoA or a precursor thereof.

This application is a continuation-in-part application of U.S. patent application Ser. No. 12/468,456, filed on May 19, 2009, which is a divisional application of U.S. patent application Ser. No. 10/637,159, filed on Aug. 8, 2003. This application is also a continuation-in-part application of U.S. patent application Ser. No. 11/466,364, filed on Aug. 22, 2006, which claims priority to U.S. Provisional Application Ser. No. 60/710,412, filed on Aug. 22, 2005. Each of the foregoing listed applications is incorporated herein by reference.

### TECHNICAL FIELD

The field of this disclosure is production of biologically active compounds by cells. More particularly, the present disclosure provides a process for increasing production of methylmalonyl-CoA and derivatives thereof.

### BACKGROUND

Despite the world's reliance on natural products from plants and microbes to treat and cure serious diseases, many fundamental questions remain to be answered as to how and why these medicines are produced in nature. Knowing more about the metabolism of these compounds will lead to simpler and more rational strategies for strain improvement. Strain improvement speeds up the drug development process and helps to reduce the cost of new drugs (Mateles, 2000).

An enormous array of medically important chemical structures are made in nature, particularly by plants and microbes. These structures fall into chemical classes based on shared routes of biosynthesis. One well-studied class of compounds is the polyketides, perhaps best characterized by the macrolide antibiotics, of which erythromycin is an example. Erythromycin and its derivatives, marketed under trade names such Biaxin®, Rulid®, and Zithromax®, are in wide use in the world today. Erythromycin's biosynthesis has been studied for over 50 years, and so it is a widely used model system for secondary metabolite production.

Like many secondary metabolites, erythromycin is a tailored polymer. The building blocks are one molecule of propionic acid and 6 molecules of methylmalonic acid in their CoA forms (Omura, 1984). Tailoring steps include the addition of two sugars, the addition of a methyl group to one sugar, and the addition of two hydroxyl groups to the polyketide polymer backbone. Despite agreement on the identity of the chemical building blocks, scientists are still unsure of the source of the propionic acid and methylmalonic acid that are used to form the molecule. Knowing this key piece of information would help lead the way to development of genetic and process manipulations in order to boost production of the antibiotic.

Originally it was reported that succinyl-CoA is the major source of methylmalonyl-CoA via the enzyme methylmalonyl-CoA mutase (MCM) (Hunaiti and Kolattukudy, 1984). Propionyl-CoA was reported to come from decarboxylation of methylmalonyl-CoA (Hsieh and Kolattukudy, 1994). These early results implied that precursors for erythromycin biosynthesis are taken at the expense of central metabolism in a reverse-anaplerotic reaction. Consistent with these results, in a different macrolide producing host, when the mutAB genes, coding for MCM, were overexpressed, a macrolide antibiotic was overproduced (Zhang et al., 1999).

Amino acid catabolism has also been identified as an important source of precursors for macrolide biosynthesis (Omura et al., 1983, 1984; Dotzlaf et al., 1984). When branched chain amino acids such as valine, isoleucine, leucine, or valine catabolites (propionate and isobutyrate) and threonine were added to the fermentation medium they boosted production of a macrolide antibiotic and its polyketide derived precursors (Omura et al., 1983, 1984, Tang et al., 1994). Conversely, when valine catabolism was blocked at the first step, (valine dehydrogenase, vdh), production of two different macrolide antibiotics went down 4-to-6-fold (Tang et al., 1994). These results pointed to amino acid catabolism, in particular branched-chain amino acid (BCAA) catabolism, as another vital source of macrolide antibiotic precursors in actinomycetes.

Surprisingly, when the BCAA catabolic pathway was blocked at a later step in propionyl-CoA carboxylase, it did not lead to a reduction in macrolide production (Donadio et al, 1996). These results conflict with those of Dotzlaf et al, (1984), but they were obtained in a different macrolide-producing host, and precursor feeding pathways have not yet been shown to operate universally in different hosts. Other workers also reported on this propionyl-CoA carboxylase reaction (Hunaiti and Kolatukuddy, 1982). Hsieh and Kolattukudy (1994) cloned a gene that recent BLASTX analyses now show could not code for a carboxylase, and may have been cloned by mistake.

Methylmalonyl-CoA mutase, coded for by the mutAB gene pair, was originally cited by Hunaiti and Kolattukudy (1984) to be the key enzyme to provide methylmalonyl-CoA for erythromycin biosynthesis. According to the conclusions of Hunaiti and Kolattukudy (1984) and Zhang et al., (1999), whose results indicated the source of methylmalonyl-CoA to be from succinyl-CoA, one would predict that a block in mutB should reduce or block production of the erythromycin. This direction for methylmalonyl-CoA mutase, though, is often referred to as the “reverse” direction, because the forward, or anaplerotic, direction towards succinyl-CoA is favored enzymatically by a factor of twenty to one (Kellermeyer, et al., 1964; Vlasie and Banerjee, 2003).

### BRIEF SUMMARY

In one aspect, the present disclosure provides a method of increasing production of a methylmalonyl-CoA derivative in a bacterium. The method includes providing a bacterium that produces a methylmalonyl-CoA derivative in which methylmalonyl-CoA mutase function has been inhibited, and culturing the bacterium in a medium containing an effective amount of methylmalonyl-CoA or a precursor thereof to increase the production of the methylmalonyl-CoA derivative by the bacterium. The production of the methylmalonyl-CoA derivative is increased when compared to production of the methylmalonyl-CoA derivative by the bacterium when cultured in a medium without the effective amount of methylmalonyl-CoA or a precursor thereof.

In another aspect, the present disclosure provides a bacterium that produces a methylmalonyl-CoA derivative and that includes a mutation in a mutB gene that inhibits methylmalonyl-CoA mutase function and increases production of the methylmalonyl-CoA derivative when cultured in a medium containing an effective amount of methylmalonyl-CoA or a precursor thereof compared to the production of the methylmalonyl-CoA derivative by the bacterium when cultured in the medium without the effective amount of methylmalonyl-CoA or a precursor thereof.

In a further aspect, a composition includes a metabolically acceptable oil, an effective amount of a substantially oil soluble methylmalonyl-CoA precursor, and at least one exogenous ingredient to sustain microbial growth.

### BRIEF DESCRIPTION OF THE DRAWINGS

FIG. 1 shows a schematic diagram of methylmalonyl-CoA metabolism;

FIG. 2 shows the effects of medium supplementation with methylmalonate and diethyl methylmalonate on FL2267 wild type and FL2281 mutB S. erythraea strains;

FIG. 3 shows the effects of stepped medium supplementation with diethyl methylmalonate from 0-25 MMA in OFM1 medium on FL2281;

FIG. 4 shows the effects of multiple dosing with diethyl methylmalonate;

FIG. 5 shows the effects of medium supplementation with methylmalonate on multiple strains;

FIG. 6 shows the effects of medium supplementation with diethyl methylmalonate on multiple strains

FIG. 7 shows the effects of oil supplementation with or without diethyl methylmalonate; and

FIG. 8 shows a TLC analysis of culture extracts.

### DETAILED DESCRIPTION

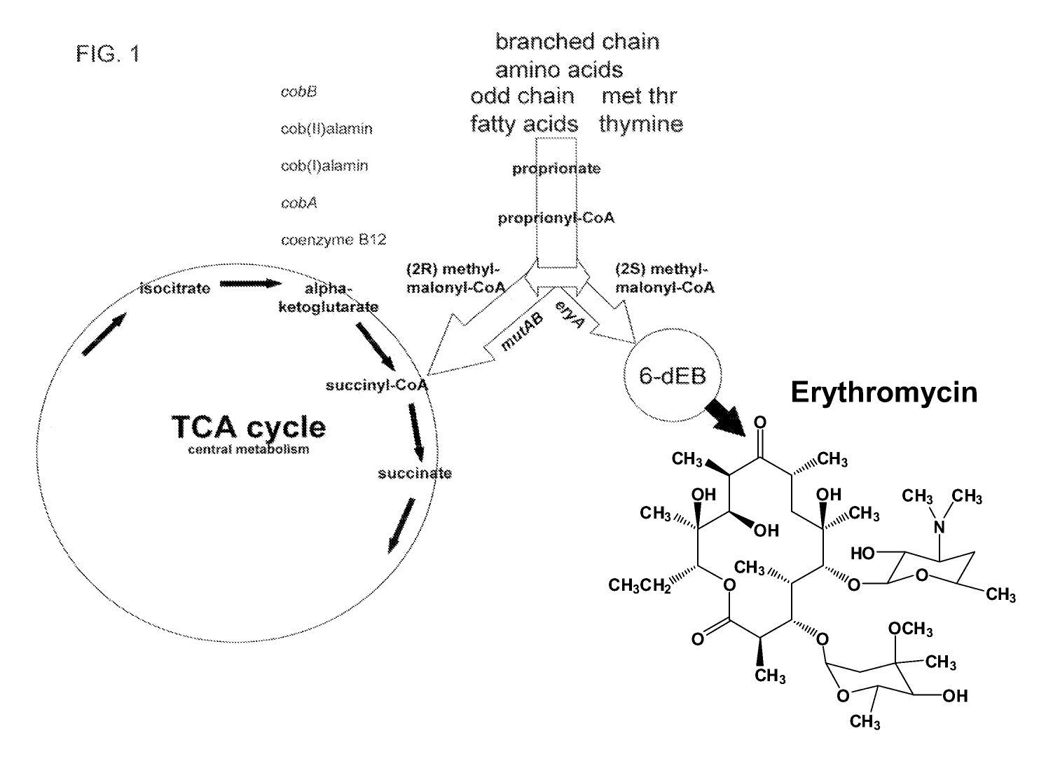

The methylmalonyl-CoA (MMCoA) metabolic node was identified previously as a target for erythromycin strain improvement manipulations in the wild type strains of Saccharopolyspora erythraea and Aeromicrobium erythreum (Reeves 2007, 2006, 2004). A schematic diagram showing such node is shown in FIG. 1. The importance of the node was originally discovered through random mutagenesis of A. erythreum followed by reverse engineering (Reeves et al., 2004). Knockouts of the mutB and cobA genes led to improved erythromycin production in carbohydrate-based medium. When tested in S. erythraea, the mutB knockout had the same positive effect on production of erythromycin in the carbohydrate medium. When an oil-based medium was used, the opposite effect was achieved, that is, the polar mutB knockout strain, FL2281, showed a 60% lower capacity for erythromycin production than the wild type strain. A metabolic model was created to explain the results in which carbon flows from succinyl-CoA to methylmalonyl-CoA in oil based medium. Strains were then created with the desire to accelerate carbon flow into the methylmalonyl-CoA metabolic node. One such construct in which the entire mutAB operon was duplicated (strain FL2385) achieved the desired result of improving erythromycin production beyond the level of the wild type strain (FL2267) by 50% (Reeves et al., 2007). However, it has now been demonstrated that inhibition of methylmalonyl-CoA mutase function counterintuitively leads to an increase of methylmalonyl-CoA derivative production.

The present disclosure describes supplementation of wild type and mutated strains of bacteria that have an inhibited methylmalonyl-CoA mutase with one or more methylmalonyl-CoA (MMCoA) precursors, such as methylmalonic acid (MMA) or diethyl methylmalonate (DiEMM) to enrich the MMCoA pool and/or increase production of MMCoA derivatives. While not wishing to be bound by theory, it is believed that DiEMM is converted to MMA through the action of esterases within a cell. Therefore, DiEMM feeds directly into the MMCoA metabolite pool. Any MMCoA precursor and combinations thereof are envisioned within the present disclosure. In one embodiment, bacteria contemplated herein include, for example, Saccharopolyspora, Aeromicrobium and Streptomyces species. In another embodiment bacteria contemplated herein include Saccharopolyspora erytlrea, Aeromicrobium erythreum, Streptomyces fradiae, Streptomyces avernitilis, Streptomyces cinnamonensis, Streptomyces antibioticus, Streptomyces venezuelae, Streptomyces violaceoniger, Streptomyces hygroscopicus, Streptomyces spp. FR-008, and Streptomyces griseus. The present disclosure further enables greater efficiency of polyketide production that may, for example, lower the cost of polyketide fermentation manufacturing. The addition of a low-cost chemical such as DiEMM may increase the production of polyketides including, for example, erythromycin, when a commercial organism is grown under conditions where the polyketide precursor MMCoA is the limiting factor for production.

The addition of MMA or DiEMM to strains that have a block in the MCM reaction, through, for example, insertion and/or deletion mutations in mutB and/or cobA, and/or reduction in co-enzyme B₁₂ availability, among other methods, also has a positive effect on the strains growth and vitality. Strains that have a block in the MCM reaction grow less vigorously and appear less healthy than the normal strain. The addition of MMA or diEMM to these cultures reverses this phenotype and makes the strain healthy again, which may lead to more efficient fermentation manufacturing through greater strain resilience to stresses related to the fermentation manufacturing process. This observation indicates that MMCoA precursors may be reversing the deleterious effects of the MCM metabolic defect in these strains.

In one embodiment according to the present disclosure, a method of increasing production of a methylmalonyl-CoA derivative in a bacterium includes providing a bacterium that produces a methylmalonyl-CoA derivative in which methylmalonyl-CoA mutase function has been inhibited and culturing the bacterium in a medium containing an effective amount of methylmalonyl-CoA or a precursor thereof to increase the production of the methylmalonyl-CoA derivative by the bacterium. The production of the methylmalonyl-CoA derivative is increased when compared to production of the methylmalonyl-CoA derivative by the bacterium when cultured in a medium without the effective amount of methylmalonyl-CoA or a precursor thereof.

In another embodiment of the method described herein, the bacterium may be selected from the group consisting of Aeromicrobium erythreum and Saccharopolyspora erythraea.

In a further embodiment of the method described herein, methylmalonyl-CoA mutase function may be inhibited by one or more of an insertion mutation or a deletion mutation of a mutB gene.

In a still further embodiment of the method described herein, the precursor may include at least one of methylmalonic acid and diethyl methylmalonate in an amount sufficient to achieve an effective amount of at least about a 1 mM concentration within the medium.

In another embodiment of the method described herein, a methylmalonyl-CoA derivative may include at least one of a polyketide or a macrolide.

In another embodiment of the present disclosure, a polyketide may be erythromycin, tylosin, niddamycin, spiramycin, oleandomycin, methymycin, neomethymycin, narbomycin, pikromycin, lankamycin, tacrolimus, rapamycin, FK520, FK506, candicidin, soraphen, ascomycin, avermectin, monensin A, or monensin B.

In a variation of the method described herein, the bacterium may be selected from the group consisting of Aeromicrobium erythreum and Saccharopolyspora erythraea, the mutation may be at least one of an insertion mutation or a deletion mutation, and the methylmalonyl-CoA derivative includes at least one of a polyketide or a macrolide

In a second embodiment of the present disclosure, a bacterium is provided that produces a methylmalonyl-CoA derivative includes a mutation in a mutB gene that inhibits methylmalonyl-CoA mutase function and increases production of the methylmalonyl-CoA derivative when cultured in a medium containing an effective amount of methylmalonyl-CoA or a precursor thereof compared to the production of the methylmalonyl-CoA derivative by the bacterium when cultured in the medium without the effective amount of methylmalonyl-CoA or a precursor thereof.

In another embodiment of the bacterium described herein, the bacterium may be selected from the group consisting of Aeromicrobium erythreum and Saccharopolyspora erythraea.

In a second embodiment of the bacterium described herein, the mutation may include at least one of an insertion mutation or a deletion mutation.

In a third embodiment of the bacterium described herein, the precursor may include at least one of methylmalonic acid and diethyl methylmalonate in an amount sufficient to achieve an effective amount of at least about a 1 mM concentration or greater within the medium.

In a fourth embodiment of the bacterium described herein, the methylmalonyl-CoA derivative may include at least one of a polyketide or a macrolide.

In a further embodiment of the bacterium described herein, the bacterium may be Saccharopolyspora erythraea, the mutation may be at least one of an insertion mutation or a deletion mutation, and the methylmalonyl-CoA derivative may inlcude at least one of a polyketide or a macrolide.

In a third embodiment of the present disclosure, a composition for culturing or fermenting a bacterium is disclosed that includes a metabolically acceptable oil, an effective amount of a substantially oil soluble methylmalonyl-CoA precursor, at least one ingredient to sustain microbial growth, and an optional buffering agent

In a further embodiment of the composition disclosed herein, a metabolically acceptable oil may include at least one of a plant oil, an animal oil, a synthetic oil, or mixtures thereof.

In another embodiment of the composition disclosed herein, an effective amount of the substantially oil soluble methylmalonyl-CoA precursor includes an amount sufficient to achieve at least about a 1 mM concentration within the composition.

In a further embodiment of the composition disclosed herein, a methylmalonyl-CoA precursor may include diethyl methylmalonate.

In another embodiment of the composition disclosed herein, an ingredient may include at least one of a monosaccharide, a disaccharide, a polysaccharide, and a corn steep liquor.

In still another embodiment of the composition disclosed herein, the composition may include a polyketide-producing bacteria.

The composition or medium for culturing or fermenting a bacterium may include any material used by a microorganism as either a carbon source, a nitrogen source, or a combined carbon-nitrogen source. In certain embodiments, a monosaccharide, a disaccharide, a polysaccharide, or a combination thereof from one or more exogenous source(s) can be added to the composition or medium. In certain embodiments at least one of glucose, fructose, sucrose, or a combination thereof may be included. Polysaccharides that may be used include, but are not limited to, soluble starch, dextrin, corn starch, and combinations thereof. In certain embodiments, monosaccharides, disaccharides, or polysaccharides may be provided in the composition or medium either individually or in aggregate at concentrations from about 1 g/L to about 60 g/L. For example, ranges of monosaccharides, disaccharides, or polysaccharides contemplated include about 1 g/L to about 2 g/L, about 1 g/L to about 4 g/L, about 2 g/L to about 5 g/L, about 5 g/L to about 10 g/L, about 10 g/L to about 15 g/L, about 20 g/L to about 30 g/L, and about 30 g/L to about 60 g/L.

In certain embodiments, a metabolically acceptable oil may include an essential oil, an organic oil, a mineral oil, a synthetic oil, and combinations thereof. Some examples include an animal oil or a plant oil which may be added to the composition or medium. Plant oils used can include, but are not limited to, coconut oil, corn oil, cottonseed oil, olive oil, palm oil, peanut oil, rapeseed oil, safflower oil, sesame oil, soybean oil, sunflower oil, and combinations or derivations thereof. A synthetic oil may be any oil made by man, a mineral oil, a modified animal or plant oil, or any non-plant or non-animal derived oil, such as a petroleum-derived oil, a hydrocarbon-based oil, chemically-based oil, and combinations thereof. Corn steep liquor may also be included in a composition or medium as a substrate for microbial growth. In certain embodiments, metabolically oils may be provided either individually or in aggregate at any of about 1 ml/L to about 60 ml/L, or about 2 ml/L to about 5 ml/L, or about 5 ml/L to about 10 ml/L, or about 10 ml/L to about 25 ml/L, or about 25 ml/L to about 60 ml/L in a composition or medium contemplated herein.

In one embodiment, buffering agents may be included in the composition or medium contemplated herein. For example, various mineral salts that include, but are not limited to, sodium chloride, calcium carbonate, magnesium sulfate, ammonium salts, including ammonium sulfate, potassium salts, phosphate salts, and/or iron sulfate may be added to the composition or medium contemplated herein. Sodium chloride may be added at a concentration of about 1 g/L to about 12 g/L (weight volume) or about 2 g/L to about 8 g/L, or about 4 g/L to about 10 g/L. Calcium carbonate may be added at a concentration of about 1 g/L to about 12 g/L (weight volume) or at about 2 g/L to about 8 g/L, or about 4 g/L to about 10 g/L. In certain embodiments, trace elements such as, for example, iron, borate, zinc, copper, molybdenum, manganese, iodine, and/or cobalt may be added to the composition or medium at concentrations conducive to microbial growth.

In another embodiment, a fermentation medium may include soybean meal, a soybean meal product, soy flour, or a soybean meal product.

In another embodiment, a composition or medium contemplated herein may be substantially free of an antimicrobial agent. In another embodiment, the composition or medium contemplated herein may include selection agents that may be used to select for specific bacteria and against other bacteria. Selection agents contemplated herein include, for example, antibiotics and/or selective nutrients, as are known in the art.

### Examples

### Example 1

### Culturing and Medium Supplementation of Bacterial Strains

OFM1 medium (Reeves et al., 2006) was used for fermentation experiments. Seed fermentations were performed in 25 ml of CFM1 medium (Reeves et al., 2006) in 250 ml shake flasks The seed cultures were incubated for 46 hrs and then 1.25 ml of seed culture was used to inoculate 25 ml of OFM1 broth in 250 ml shake flasks fermentations that were incubated at 32° C. at 380 rpm and 65% humidity for 120 hrs. The medium supplements of methylmalonic acid (MMA, Aldrich M54058) and diethyl methylmalonate (DiEMM; Aldrich #126136) were added at a concentration of 10 mM, unless otherwise indicated. These same general conditions were followed in subsequent examples, unless otherwise indicated.

### Example 2

### Processing of Fermentation Samples for Bioassays and TLC

Cells were removed from broth samples by centrifugation, and each supernatant was passed through a 0.45-μm filter. A 50-μl portion of the filtered supernatant was removed for bioassay analysis, and the remainder of the supernatant was used for further analysis by thin-layer chromatography (TLC). The remaining supernatant (700 μl) was extracted with 500 μl of ethyl acetate. The extract was concentrated to dryness, and the extract was then resuspended in 100 μl of acetonitrile. A 10-μl portion of the acetonitrile solution was used for analysis of polyketide production by TLC. The solvent used for visualization included isopropylether, chloroform, ammonium hydroxide (75:35:1) and an anisaldehyde spray detection reagent.

Bioassays were performed to determine the erythromycin yields in culture broth, and the methods used have been described previously (Reeves et al.).

### Example 3

### Effects of Medium Supplementation with methylmalonate and diethyl methylmalonate

It had been previously reported that the mutB knockout strain, FL2281, showed a significant reduction in erythromycin production (Reeves et al., 2006) in an oil-based fermentation medium (OFM1). In this study, the deficiency in erythromycin production is overcome and turned into a surplus of production through the addition of 10 MMA DiEMM. Addition of the unesterified precursor MMA also allows the deficiency to be partially overcome.

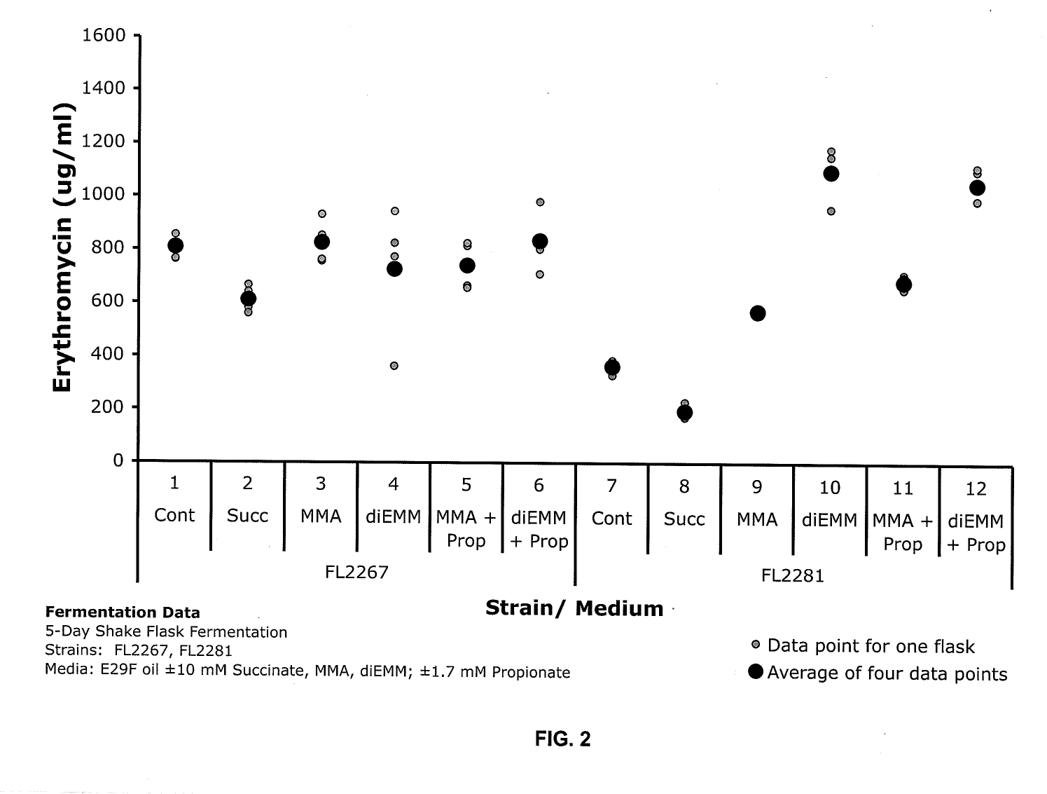

OFM1 medium was supplemented with 10 mM methylmalonate (MM) to see whether it would stimulate erythromycin production as predicted by the metabolic model (Reeves et al., 2007). Three other metabolites were also added to the fermenation as controls: diethyl methylmalonate (DiEMM), a much less expensive ester-derivative of MM and succinate and propionate, two other closely related metabolites. Two different strains were used in this experiment, the S. erythraea wild type strain FL2267, and a S. erythraea mutB knockout strain, FL2281 having a reduction or inhibition of MMCoA mutase function. The results (FIG. 2) show that the mutB knockout strain FL2281 was responsive to MMA supplementation and unexpectedly highly responsive to DiEMM supplementation. The FL2281 strain showed a 60% increase in erythromycin production with the MMA supplementation and a 200% increase with DiEMM supplementation. The 200% increase in production represented a 70% molar conversion of DiEMM to erythromycin, assuming seven DiEMM molecules are consumed by the polyketide synthase to form one erythromycin molecule. This is an unexpected conversion efficiency considering the complexity of the erythromycin biosynthesis process, and the theoretical potential for DiEMM to be metabolized through other pathways of intermediary metabolism. These results are contrary to an experiment where mutB strains were shown to be unable to grow on MMA as a sole carbon source (Reeves et al., 2006) indicating that MMA is not available to intermediary metabolism but is solely used for secondary metabolite production. Neither MMA nor DiEMM boosted erythromycin production by the wild type strain, FL2267, at these concentrations. Succinate had the effect of reducing production levels in FL2281 and FL2267 by 50% and 25%, respectively. The addition of propionate to the MM and DiEMM showed no additional significant effect on production, as might be expected due to its role in biosynthesis.

The present example illustrates the utility of supplementation of oil-containing industrial fermentation media with MMA and more importantly with DiEMM for polyketide synthesis. These results further demonstrate considerable economic potential of supplementation with DiEMM, which is considerably less expensive than MMA or MMCoA at quantities needed for industrial scale production, to drive polyketide production in industrial scale fermentations. Moreover, precursor feeding may be a viable alternative to metabolic engineering when a strain is operating below its maximal potential output. Still further, precursor feeding along with metabolic engineering may create new rates of production for industrial strains. These results indicate that supplementation with a polyketide precursor, such as DiEMM, removes MMCoA availability as a limiting factor for production polyketide synthesis.

### Example 4

### Medium Supplementation with diethyl methylmalonate from 0-25 MMA in OFM1 Medium

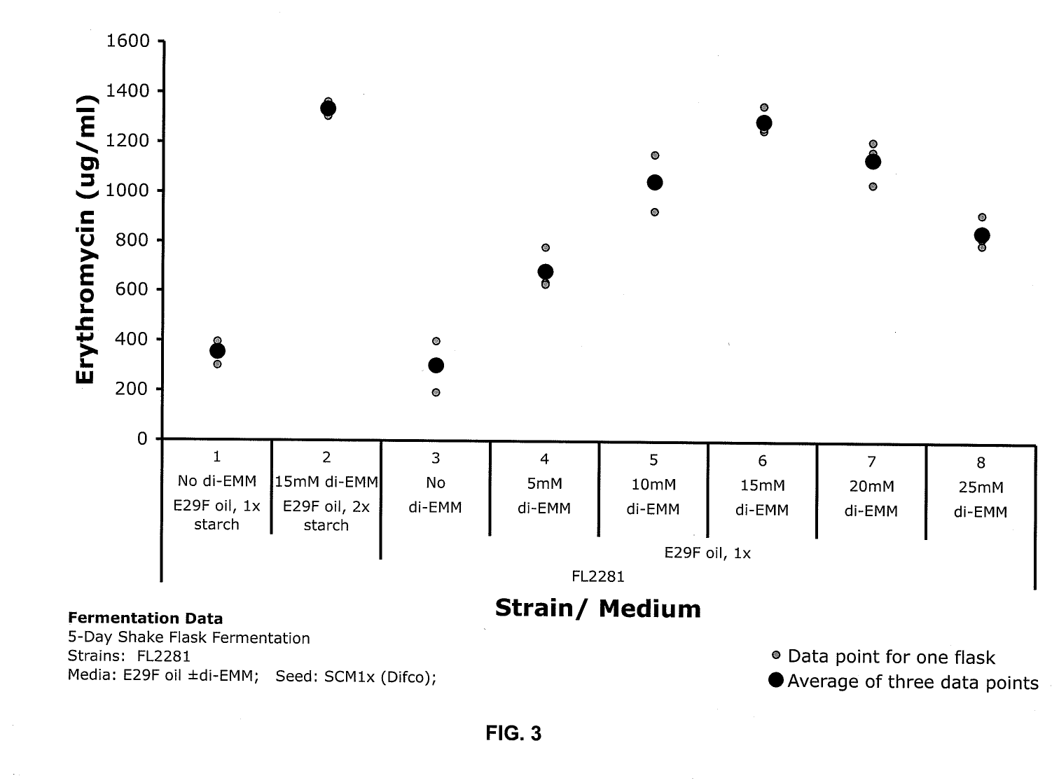

Based on the results from the previous experiment, supplementation of OFM1 medium with various levels of DiEMM was performed in order to find the concentration of DiEMM that had the maximal stimulatory effect. The initial results (FIG. 3) showed that DiEMM stimulatory effect was maximal at 15 mM; above that level production levels began to drop-off significantly. Even the lowest level of DiEMM supplementation of 5 mM showed a significant stimulation of erythromycin production. The conversion efficiencies of DiEMM to erythromycin were highest at the 5 mM MMA and 10 mM MMA levels and as the DiEMM concentration went above 10 mM MMA the efficiency of conversion began to level off. While these results demonstrate that 5 mM MMA and 10 mM MMA levels are effective amounts for increasing polyketide synthesis, it is believed that amounts as low as about 1 mM MMA will increase polyketide synthesis.

### Example 5

### Multiple Dosing of diethyl methylmalonate

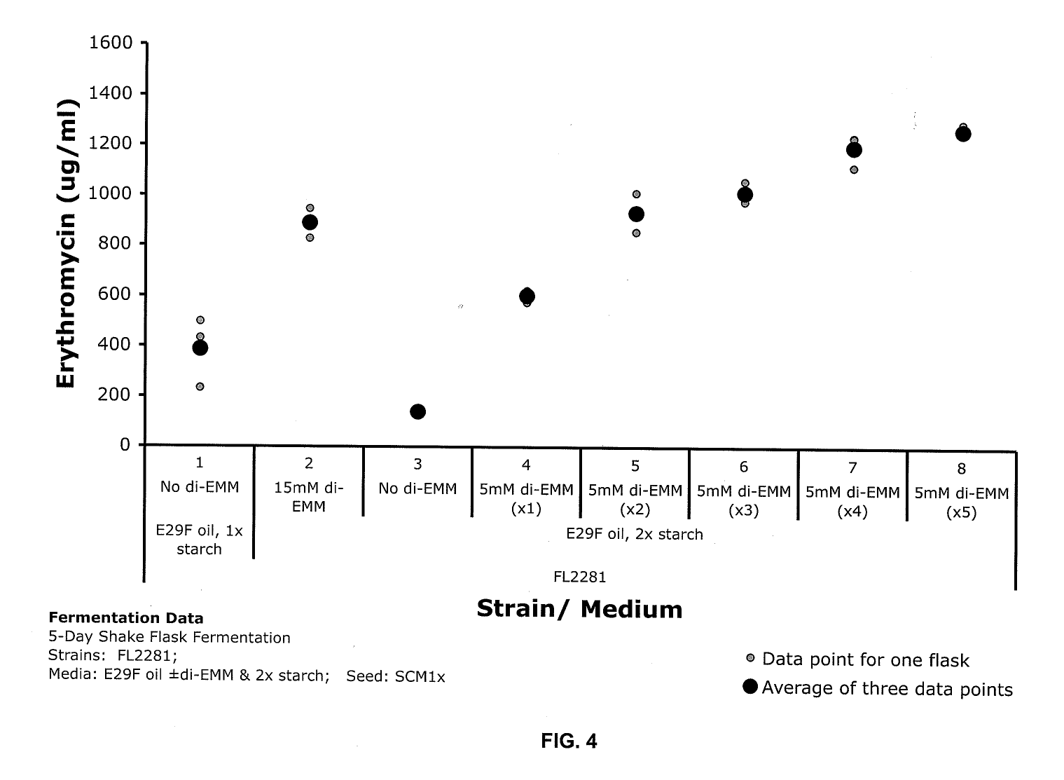

Supplementation of OFM1 medium with DiEMM in multiple smaller doses, instead of adding DiEMM all at once in the beginning of the fermentation, was tested next (FIG. 4). Using this approach a larger total amount of DiEMM (25 mM) was needed to achieve the same absolute yield increase achieved on a single dose basis at 15 mM. Furthermore, the efficiency of conversion of DiEMM to erythromycin decreased as the fermentation progressed, so that by the time the fifth dose was added, the resulting effect on erythromycin production was nearly undetectable.

### Example 6

### Effects of methylmalonate in OFM1 Medium on Multiple Strains

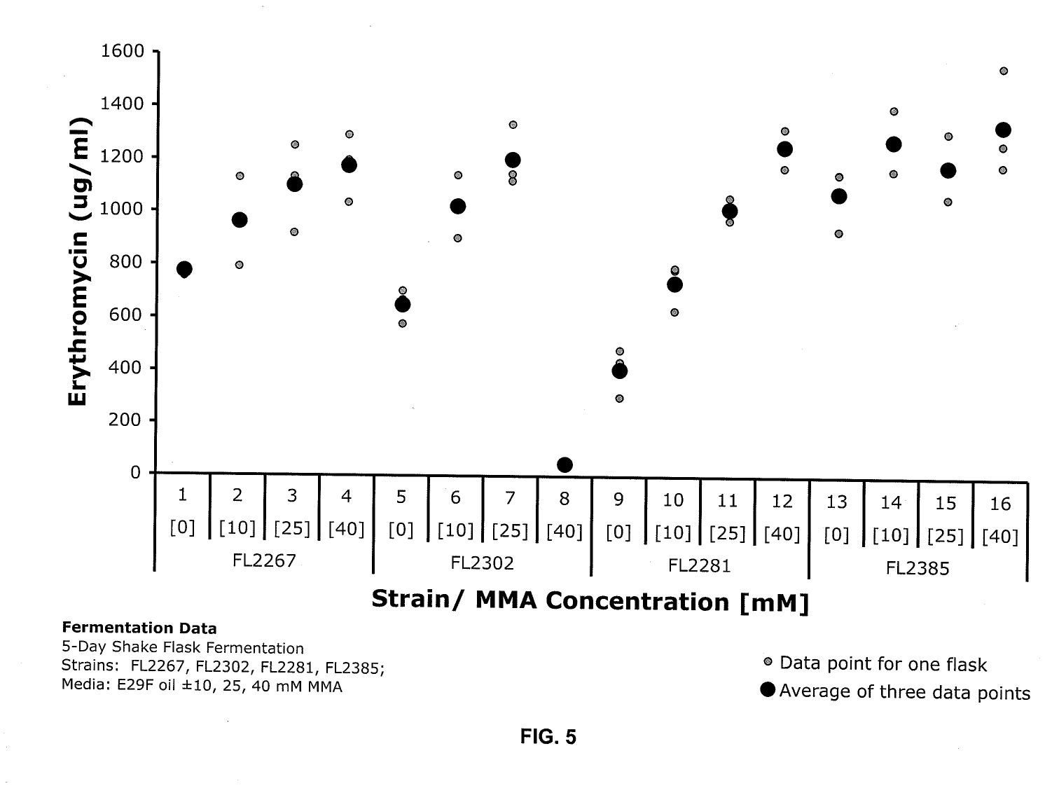

When additional strains, including FL2302 (an in frame mutB mutation) and FL2385 (a mutAB operon duplication strain) were tested, MMA supplementation at varying concentrations obtained similar results (FIG. 5). The MMA tended to stimulate production of erythromycin by all strains. These results demonstrate that various ways of inhibiting MMCoA mutase function through mutB mutations are effective at isolating polyketide production from the TCA cycle, effectively causing a short circuit in the MMCoA metabolic node, in that, MMCoA precursors are shunted directed to polyketide synthesis and are not lost to general metabolism via the citric acid cycle. This conclusion is supported by the observation that MMA and DiEMM added at the beginning of the fermentation persist in the fermentation medium over the course of the fermentation and are not immediately broken down and used for metabolic energy. Combining supplementation of MMCoA precursors with bacterial or other strains in which general metabolism has been separated from polyketide synthesis pathways, such that all metabolites for polyketide synthesis are only used for polyketide production may significantly increase polyketide production efficiency.

### Example 7

### Effects of diethyl methylmalonate in OFM1 Medium on Multiple Strains

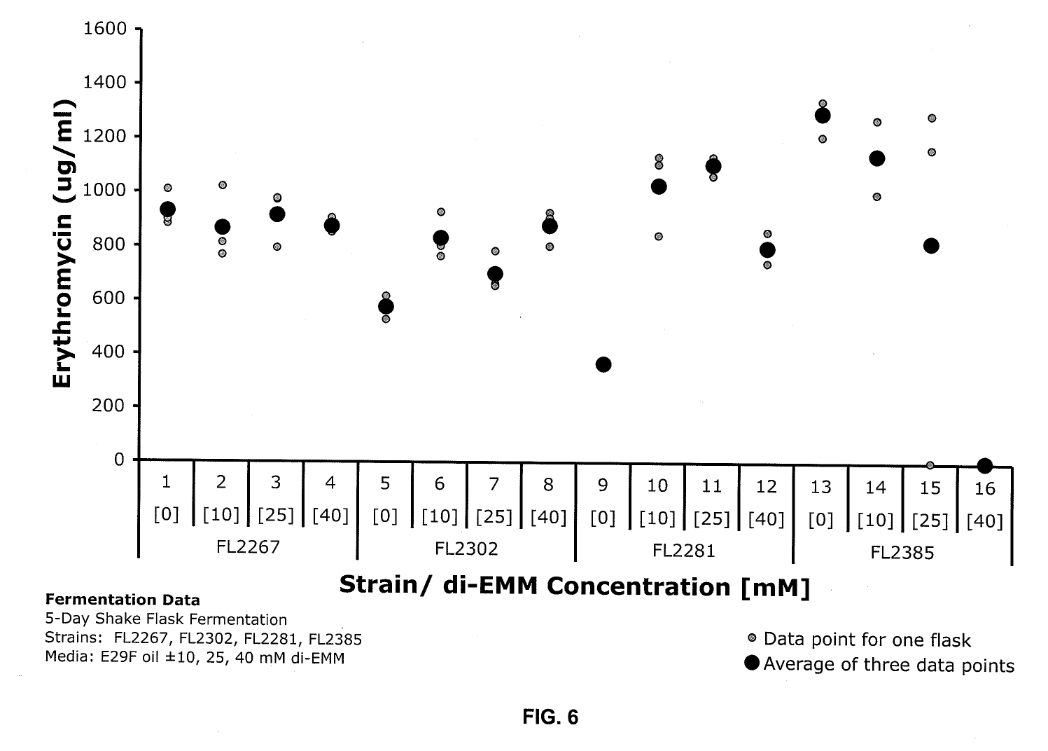

Comparison among different strains of the effects of supplementation of DiEMM at different concentrations showed that wild type was unaffected by supplementation (FIG. 6). Both mutB knockouts, FL2302 and FL2281 exhibited boosts in erythromycin production, with the more notable results for the FL2281 strain. FL2385 is hurt by diEMM addition and makes no erythromycin at 40 mM diEMM despite showing growth, and shows enhanced production in 0 diEMM over all other strains.

### Example 8

### Effects of Oil Supplementation with or without diethyl methylmalonate

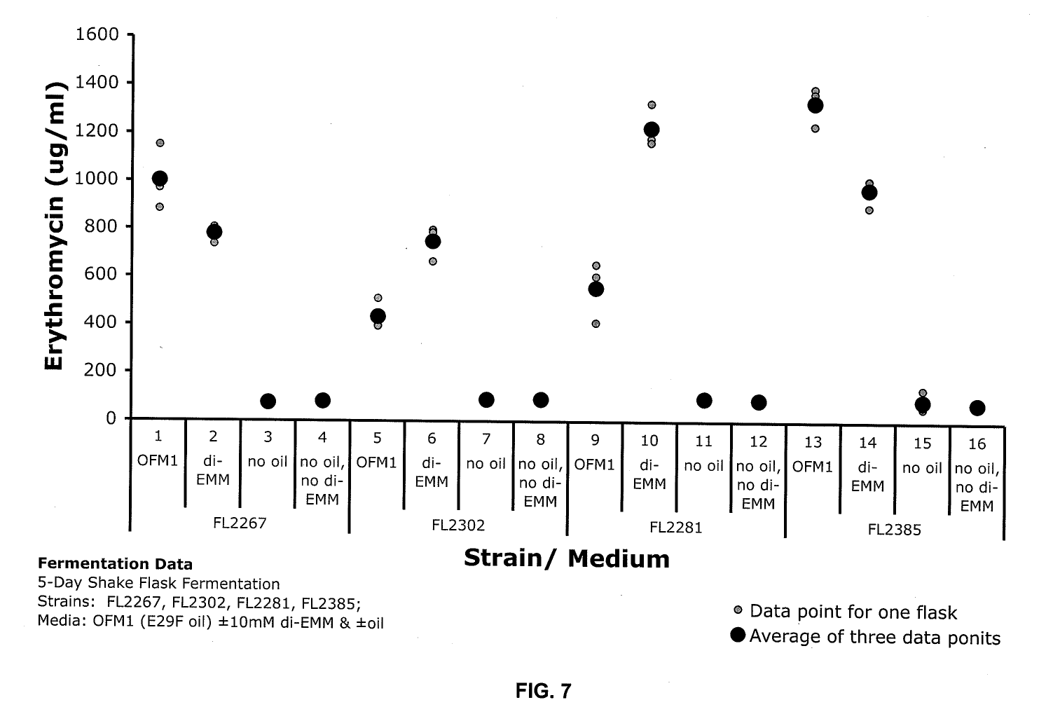

The extent of DiEMM effect on various strains was investigated in media with or without oil (FIG. 7). DiEMM addition consistently increases FL2302 and FL2281 production off erythromycin with FL2281 responding the most. FL2267 and FL2385 respond negatively to DiEMM. All strains required oil for growth and erythromycin production, but mostly for erythromycin production. Moreover, DiEMM supplementation does not eliminate the need for oil in the medium.

DiEMM has limited solubility in water but is substantially soluble in oil such that oil is an effective carrier of DiEMM in a medium allowing at least an effective amount of DiEMM to be added to the medium. Any means that would increase the ability of DiEMM to be dispersed in media are contemplated herein, such as through the use of one or more emulsions among other means. For example, bacteria could be cultured in an oil based medium or a medium having a minimal amount of oil sufficient to deliver the DiEMM to the culture. This minimum amount can be determine by incremental supplementation of oil to a non-oil containing medium until an increase in polyketide synthesis is observed when other relevant medium components are not limiting. Further, it is believed that oil or oil-like media may serve as a reservoir for the DiEMM, therefore supplementation of media with oil containing DiEMM may increase efficiency of the fermentation process by reducing the number of steps for introducing the DiEMM. Further, to guard against reaching a saturation point of DiEMM in the fermentation media, excess oil may be added to ensure adequate solubility of the DiEMM at any effective amount added.

### Example 9

### TLC Analysis of Culture Extracts

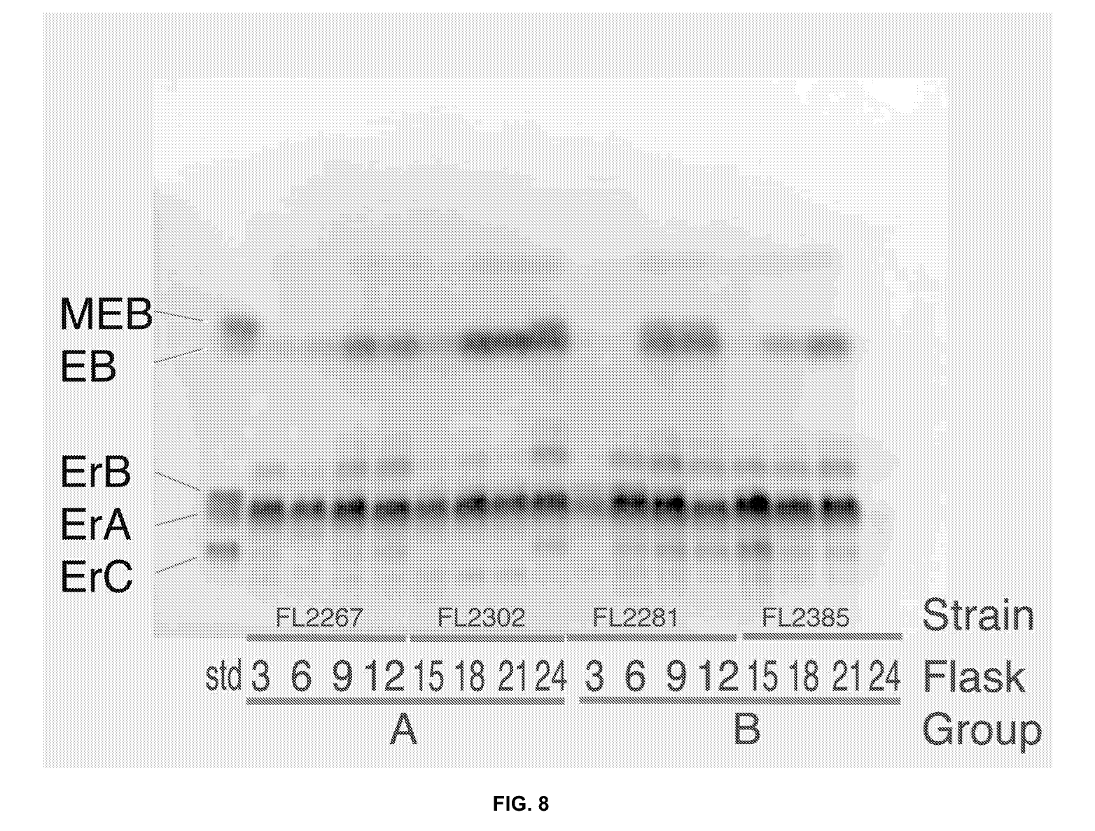

Insight into the mechanism responsible for the plateauing effect of erythromycin production was sought through thin chromatographic analysis (TLC) of extracts from culture broths (FIG. 8). FIG. 8 illustrates a TLC chromatograph of solvent extracts from S. erythraea fermentations in OFM1 broth supplemented with DiEMM at 0, 10, 25, and 40 mM concentrations. Shown are results from wild type S. erythraea (FL2267), two mutB knockout strains (FL2302 and FL2281) and one MCM operon duplication strain (FL2385).

The chromatograph indicates that in addition to the expected erythromycin species A (ErA), B (ErB), and C (ErC), the polyketides EB (erythronolide B) and MEB (3-alpha-mycarosylerythronolide B) are also apparent in our screen. These results reveal that even more extensive stimulation of polyketide production occurs in supplemented broth as evidenced by the combined production of both EB, an early stage polyketide intermediate, and ErA. This new information reflects not only the power of medium supplementation with the right precursor to stimulate yield (other metabolites such as propionate or succinate did not stimulate yield), but also revealed that methylmalonyl-CoA appears to not remain the limiting factor for erythromycin A production. Therefore, these results indicate that supplementing fermentation media with MMCoA or precursors thereof for production of polyketides in bacteria with inhibited methylmalonyl-CoA mutase function is an effective way to increase polyketide synthesis, as under such conditions, MMCoA supply appears to no longer be a limiting factor in polyketide production.

The present disclosure is representational of certain concepts that may be employed to increase methylmalonyl-CoA or a derivative thereof, the drawings and description herein detail specific examples and embodiments thereof. From the foregoing, it will be observed that numerous variations and modifications may be effected without departing from the spirit and scope of the disclosure. It is to be understood that no limitation with respect to the specific disclosure herein is intended or should be inferred. All patents, patent applications, and other references disclosed herein are incorporated by reference.

### REFERENCES

Aparicio J F, Caffrey P, Gil J A. Zotchev S B. Polyene antibiotic biosynthesis gene clusters. Appl Microbiol Biotechnol. 2003 May; 61(3):179-88. Epub 2002 Dec. 18.
Aparicio J F, Molnar I, Schwecke T. Konig A, Haydock S F, Khaw L E, Staunton J, Leadlay P F. Organization of the biosynthetic gene cluster for rapamycin in Streptomyces hygroscopicus: analysis of the enzymatic domains in the modular polyketide synthase. Gene. 1996 Feb. 22; 169(1):9-16.
Bibb M J, White J, Ward J M, Janssen G R. The mRNA for the 23S rRNA methylase encoded by the ermE gene of Saccharopolyspora erythraea is translated in the absence of a conventional ribosome-binding site. Mol Microbiol. 1994 November; 14(3):533-45.
Birch A, Leiser A, Robinson J A. Cloning, sequencing, and expression of the gene encoding methylmalonyl-coenzyme A mutase from Streptomyces cinnamonensis. J Bacteriol. 1993 June; 175(11):3511-9.
Dayem L C, Carney J R, Santi D V, Pfeifer B A, Khosla C. Kealey J T. Metabolic engineering of a methylmalonyl-CoA mutase-epimerase pathway for complex polyketide biosynthesis in Escherichia coli. Biochemistry. 2002 Apr. 23; 41(16):5193-201.
Donadio S, Stayer M J, Katz L. Erythromycin production in Saccharopolyspora erythraea does not require a functional propionyl-CoA carboxylase. Mol Microbiol. 1996 March; 19(5):977-84.
Dotzlaf J E, Metzger L S, Foglesong M A. Incorporation of amino acid-derived carbon into tylactone by Streptomyces fradiae GS 14. Antimicrob Agents Chemother. 1984 February; 25(2):216-20.
Fleischmann R D, Adams M D, White 0, Clayton R A, Kirkness E F, Kerlavage A R, Bult C J, Tomb J F, Dougherty B A, Merrick J M, et al. Whole-genome random sequencing and assembly of Haemophilus influenzae Rd. Science. 1995 Jul. 28; 269(5223):496-512.
Gerth K, Bedorf N, Irschik H, Hofle G, Reichenbach H. The soraphens: a family of novel antifungal compounds from Sorangium cellulosum (Myxobacteria). I. Soraphen Al alpha: fermentation, isolation, biological properties. J Antibiot (Tokyo). 1994 January; 47(1):23-31.
Gil J A. Campelo-Diez A B. Candicidin biosynthesis in Streptomyces griseus. Appl Microbiol Biotechnol. 2003 February; 60(6):633-42. Epub 2002 Dec. 18. Review.
Goryshin, I. Y., and W. S. Reznikoff. 1998. Tn5 in vitro transposition. J. Biol. Chem. 273: 7367-7374.
Haydock S F, Aparicio J F. Molnar 1, Schwecke T, Khaw L E, Konig A, Marsden A F, Galloway I S, Staunton J. Leadlay P F. Divergent sequence motifs correlated with the substrate specificity of (methyl)malonyl-CoA:acyl carrier protein transacylase domains in modular polyketide synthases. FEBS Lett. 1995 Oct. 30; 374(2):246-8.
Hsieh Y J, Kolattukudy P E. Inhibition of erythromycin synthesis by disruption of malonyl-coenzyme A decarboxylase gene eryM in Saccharopolyspora erythraea. J Bacteriol. 1994 February; 176(3):714-24.
Hunaiti A A, Kolattukudy P E. Source of methylmalonyl-coenzyme A for erythromycin synthesis: methylmalonylcoenzyme A mutase from Streptomyces erythreus. Antimicrob Agents Chemother. 1984 February; 25(2):173-8.
Hu Z, Bao K. Zhou X, Zhou Q, Hopwood D A, Kieser T. Deng Z. Repeated polyketide synthase modules involved in the biosynthesis of a heptaene macrolide by Streptomyces sp. FR-008. Mol Microbiol. 1994 October; 14(1):163-72.
Ikeda H, Nonomiya T, Usami M. Ohta T. Omura S. Organization of the biosynthetic gene cluster for the polyketide anthelmintic macrolide avermectin in Streptomyces avermitilis. Proc Natl Acad Sci USA. 1999 Aug. 17; 96(17):9509-14.
Keiser, T., Bibb, M. J., Buttner, M. J., Chater, K. F. & Hopwood, D. A. Practical Streptomyces Genetics (John Innes Foundation, Norwich, 2000.
Kellermeyer R W, Allen S H G, Stjernholm R, and Wood H G. Methylmalonyl isomerase. IV. Purification and properties of the enzyme from Propionibacteria. J. Biol. Chem. 1964 239:2562-2569
Liu 1-1, Reynolds K A. Role of crotonyl coenzyme A reductase in determining the ratio of polyketides monensin A and monensin B produced by Streptomyces cinnamonensis. J Bacteriol. 1999 November; 181(21):6806-13.
F Lombó, B Pfeifer, T Leaf, S Ou, Y S Kim, D E Cane, P Licari, C Khosla. Enhancing the atom economy of polyketide biosynthetic processes through metabolic engineering. Biotechnol Prog. 2001 vol. 17 (4) pp. 612-7
Marsh E N, McKie N, Davis N K, Leadlay P F. Cloning and structural characterization of the genes coding for adenosylcobalamin dependent methylmalonyl-CoA mutase from Propionibacterium she rmanii. Biochem J. 1989 Jun. 1; 260(2):345-52.
Mateles, Richard I. Penicillin: A Paradigm for Biotechnology Candida Corporation, Illinois, ISBN 1 891545 01 9
Miller E S. Cloning vectors, mutagenesis, and gene disruption (ermR) for the erythromycin-producing bacterium Aeromicrobium erythreum. Appl Environ Microbiol. 1991 September; 57(9):2758-61
Mochizuki S. Hiratsu K, Suwa M. Ishii T. Sugino F. Yamada K. Kinashi H. The large linear plasmid pSLA2-L of Streptomyces rochei has an unusually condensed gene organization for secondary metabolism. Mol Microbiol. 2003 June; 48(6):1501-10.
Molnar I, Aparicio J F, Haydock S F, Khaw L E, Schwecke T, Konig A, Staunton J. Leadlay P F. Organisation of the biosynthetic gene cluster for rapamycin in Streptomyces hygroscopicus: analysis of genes flanking the polyketide synthase. Gene. 1996 Feb. 22; 169(1):1-7.
Oh S H, Chater K F. Denaturation of circular or linear DNA facilitates targeted integrative transformation of Streptomyces coelicolor A3(2): possible relevance to other organisms. J Bacteriol. 1997 January; 179(1):122-7.
Omura S, Tsuzuki K, Tanaka Y, Sakalcibara H, Aizawa M, Lukacs G. Valine as a precursor of n-butyrate unit in the biosynthesis of macrolide aglycone. J Antibiot (Tokyo). 1983 May; 36(5):614-6.
Omura S, Taki A, Matsuda K, Tanaka Y. Ammonium ions suppress the amino acid metabolism involved in the biosynthesis of protylonolide in a mutant of Streptomyces fradiae. J Antibiot (Tokyo). 1984 November; 37(11):1362-9.
Omura, S., Macrolide Antibiotics: Chemistry, Biology, Practice, Academic Press, New York, 1984.
Omura S, Takeshima H. Nakagawa A. Miyazawa J, Piriou F, Lukacs G. Studies on the biosynthesis of 16-membered macrolide antibiotics using carbon-13 nuclear magnetic resonance spectroscopy. Biochemistry. 1977 Jun. 28; 16(13):2860-6.
Paulus Ti, Tuan J S, Luebke V E, Maine G T, DeWitt J P, Katz L. Mutation and cloning of eryG, the structural gene for erythromycin 0-methyltransferase from Saccharopolyspora erythraea, and expression of eryG in Escherichia coli. J Bacteriol. 1990 May; 172(5):2541-6.
Reeves A R, Weber G, Cernota W H, Weber J M. Analysis of an 8.1-kb DNA fragment contiguous with the erythromycin gene cluster of Saccharopolyspora erythraea in the eryCI-flanking region. Anti microb Agents Chemother. 2002 December; 46(12):3892-9.
Reeves A R, Igor A Brikun, William H Cernota, Benjamin I Leach, Melissa C Gonzalez, J. Mark Weber. Effects of methylmalonyl-CoA mutase gene knockouts on erythromycin production in carbohydrate-based and oil-based fermentations of Saccharopolyspora erythraea. J IND MICROBIOL BIOTECHNOL (2006) 33: 600-609.
Reeves A R, Brikun I A, Cernota W H, Leach B I, Gonzalez M C, Weber J M. Engineering of the methylmalonyl-CoA metabolite node of Saccharopolyspora erythraea for increased erythromycin production. Metab Eng. 2007 May; 9(3):293-303.
Rodicio M R, Chater K R Small DNA-free liposomes stimulate transfection of streptomyces protoplasts. J Bacteriol. 1982 September; 151(3):1078-85.
Roberts A N, Barnett L, Brenner S. Transformation of Arthrobacter and studies on the transcription of the Arthrobacter ermA gene in Streptomyces lividans and Escherichia coli. Biochem J. 1987 Apr. 15; 243(2):431-6.
Rodriguez L. Aguirrezabalaga I. Allende N. Brana A F, Mendez C. Salas J A. Engineering deoxysugar biosynthetic pathways from antibiotic-producing microorganisms. A tool to produce novel glycosylated bioactive compounds. Chem Biol. 2002 June; 9(6):721-9.
Sambrook, J., Fritsch, E. F., and Maniatis, T., (1989) “Molecular Cloning”, Cold Spring Harbour Laboratory Press, ISBN 0-87969-309-6.
Schwecke T. Aparicio J F, Molnar I. Konig A. Khaw L E, Haydock S F, Oliynyk M, Caffrey P. Cortes J, Lester J B, et al. The biosynthetic gene cluster for the polyketide immunosuppressant rapamycin. Proc Natl Acad Sci USA. 1995 Aug. 15; 92(17):7839-43.
Smith D B, Johnson K S. Single-step purification of polypeptides expressed in Escherichia coli as fusions with glutathione S-transferase. Gene. 1988 Jul. 15; 67(1):31-40.
Tang L, Zhang Y X, Hutchinson C R. Amino acid catabolism and antibiotic synthesis: valine is a source of precursors for macrolide biosynthesis in Streptomyces ambofaciens and Streptomyces fradiae. J Bacteriol. 1994 October; 176(19):6107-19.
Vlasie M D, Banerjee R. Tyrosine 89 accelerates Co-carbon bond homolysis in methylmalonyl-CoA mutase. J Am Chem Soc. 2003 May 7; 125(18):5431-5.
Ward J M, Janssen G R, Kieser T, Bibb M J, Buttner M J, Bibb M J. Construction and characterisation of a series of multi-copy promoter-probe plasmid vectors for Streptomyces using the aminoglycoside phosphotransferase gene from Tn5 as indicator. Mol Gen Genet. 1986 June; 203(3):468-78.
Weber J M, Wierman C K, Hutchinson C R. Genetic analysis of erythromycin production in Streptomyces erythreus. J Bacteriol. 1985 October; 164(1):425-33.
Weber J M, Leung J O, Maine G T, Potenz R H, Paulus T J, DeWitt J P. Organization of a cluster of erythromycin genes in Saccharopolyspora erythraea. J Bacteriol. 1990 May; 172(5):2372-83.
Weber J M, Leung J O, Swanson S J, Idler K B, McAlpine J B. An erythromycin derivative produced by targeted gene disruption in Saccharopolyspora erythraea. Science. 1991 Apr. 5; 252(5002):114-7.
Wu K, Chung L, Revill W P, Katz L, Reeves C D. The FK520 gene cluster of Streptomyces hygroscopicus var. ascomyceticus (ATCC 14891) contains genes for biosynthesis of unusual polyketide extender units. Gene. 2000 Jun. 13; 251(1):81-90.
Xue Y. Wilson D, Sherman D H. Genetic architecture of the polyketide synthases for methymycin and pikromycin series macrolides. Gene. 2000 Mar. 7; 245(1):203-11.
Watson et al. Molecular Biology of the Gene, 4th Edition, 1987, The Bejacmin/Cummings Pub. co., p. 224).
Zhang W, Yang L, Jiang W, Zhao G, Yang Y, Chiao J. Molecular analysis and heterologous expression of the gene encoding methylmalonylcoenzyme A mutase from rifamycin SV-producing strain Amycolatopsis mediterranei U32. Appl Biochem Biotechnol. 1999 December; 82(3):209-25.

1. A method of increasing production of a methylmalonyl-CoA derivative in a bacterium, the method comprising
providing a bacterium that produces a methylmalonyl-CoA derivative in which methylmalonyl-CoA mutase function has been inhibited; and
culturing the bacterium in a medium containing an effective amount of methylmalonyl-CoA or a precursor thereof to increase the production of the methylmalonyl-CoA derivative by the bacterium,
wherein the production of the methylmalonyl-CoA derivative is increased when compared to production of the methylmalonyl-CoA derivative by the bacterium when cultured in a medium without the effective amount of methylmalonyl-CoA or a precursor thereof.

2. The method of claim 1, wherein the bacterium is selected from the group consisting of Aeromicrobium erythreum and Saccharopolyspora erythraea.

3. The method of claim 2, wherein methylmalonyl-CoA mutase function has been inhibited by one or more of an insertion mutation or a deletion mutation of a mutB gene.

4. The method of claim 3, wherein the precursor comprises at least one of methylmalonic acid and diethyl methylmalonate in an amount sufficient to achieve an effective amount of at least about a 1 mM concentration within the medium.

5. The method of claim 1, wherein the methylmalonyl-CoA derivative comprises at least one of a polyketide or a macrolide.

6. The method of claim 5, wherein the polyketide is selected from the group consisting of erythromycin, tylosin, niddamycin, spiramycin, oleandomycin, methymycin, neomethymycin, narbomycin, pikromycin, lankamycin, tacrolimus, rapamycin, FK520, FK506, candicidin, soraphen, ascomycin, avermectin, monensin A, and monensin B.

7. The method of claim 1, wherein the bacterium is selected from the group consisting of Aeromicrobium erythreum and Saccharopolyspora erythraea, the mutation is at least one of an insertion mutation or a deletion mutation, and the methylmalonyl-CoA derivative comprises at least one of a polyketide or a macrolide.

8. A bacterium that produces a methylmalonyl-CoA derivative comprising a mutation in a mutB gene that inhibits methylmalonyl-CoA mutase function and increases production of the methylmalonyl-CoA derivative when cultured in a medium containing an effective amount of methylmalonyl-CoA or a precursor thereof compared to the production of the methylmalonyl-CoA derivative by the bacterium when cultured in the medium without the effective amount of methylmalonyl-CoA or a precursor thereof.

9. The bacterium of claim 8, wherein the bacterium is selected from the group consisting of Aeromicrobium erythreum and Saccharopolyspora erythraea.

10. The bacterium of claim 9, wherein the mutation comprises at least one of an insertion mutation or a deletion mutation.

11. The bacterium of claim 10, wherein the precursor comprises at least one of methylmalonic acid and diethyl methylmalonate in an amount sufficient to achieve an effective amount of at least about a 1 mM concentration within the medium.

12. The bacterium of claim 8, wherein the methylmalonyl-CoA derivative comprises at least one of a polyketide or a macrolide.

13. The bacterium of claim 12, wherein the polyketide is selected from the group consisting of erythromycin, tylosin, niddamycin, spiramycin, oleandomycin, methymycin, neomethymycin, narbomycin, pikromycin, lankamycin, tacrolimus, rapamycin, FK520, FK506, candicidin, soraphen, ascomycin, avermectin, monensin A, and monensin B.

14. The bacterium of claim 11, wherein the bacterium comprises Saccharopolyspora erythraea, the mutation is at least one of an insertion mutation or a deletion mutation, and the methylmalonyl-CoA derivative comprises at least one of a polyketide or a macrolide.

15. A composition for culturing or fermenting a bacteria comprising:
a metabolically acceptable oil,
an effective amount of a substantially oil soluble methylmalonyl-CoA precursor;
at least one ingredient to sustain microbial growth; and
an optional buffering agent.

16. The composition of claim 15, wherein the metabolically acceptable oil comprises at least one of a plant oil, an animal oil, a synthetic oil, or mixtures thereof.

17. The composition of claim 16, wherein the effective amount of the substantially oil soluble methylmalonyl-CoA precursor comprises an amount sufficient to achieve at least about a 1 mM concentration within the composition.

18. The composition of claim 17, wherein the methylmalonyl-CoA precursor comprises diethyl methylmalonate.

19. The composition of claim 18, wherein the at least one ingredient comprises at least one of a monosaccharide, a disaccharide, a polysaccharide, and a corn steep liquor.

20. The composition of claim 19, further comprising a polyketide-producing bacteria.
